(19) United States
(12) Patent Application Publication
(10) Pub. No.: US 2021/0196294 A1
(43) Pub. Date: Jul. 1, 2021

(54) ULTRASONIC DEVICE

(71) Applicant: Nicolas Aeby, Geneva (CH)

(72) Inventor: Nicolas Aeby, Geneva (CH)

(21) Appl. No.: 17/198,409

(22) Filed: Mar. 11, 2021

Related U.S. Application Data
(63) Continuation-in-part of application No. PCT/IB2018/056926, filed on Sep. 11, 2018.

(51) Int. Cl. A61B 17/22

(52) U.S. Cl. CPC A61B 17/2202; A61B 2017/22038

(57) ABSTRACT

An ultrasonic device for treatment of atherosclerosis. The device includes a sonotrode, a transmission wire and a tip, said sonotrode comprising a transducer and a horn, the transducer being coupled to the horn. The transmission wire is coupled on one end with the horn and on the opposite end with the tip, so that when the transducer vibrates, said vibration are amplified by the horn and transmitted to the transmission wire that displaces accordingly, said displacements of the wire inducing vibrations of the tip generating an acoustic field from said tip. The device comprises n sonotrode, n transmission wire, and at least one tip, n being superior or equal to two. The device further comprises a control module being arranged for controlling the amplitude of displacement of each transmission wire so as to set up the emitted acoustic field generated from said tip.

### RELATED APPLICATIONS

This application is a continuation-in-part of International Application No. PCT/IB2018/056926, filed Sep. 11, 2018 and published as International Publication No. WO 2020/053624, the disclosure of which is incorporated by reference herein in its entirety.

### FIELD OF THE DISCLOSURE

The present disclosure is directed to generally to an ultrasonic device and more specifically to an ultrasonic device for the treatment of atherosclerosis.

### BACKGROUND OF THE DISCLOSURE

Ultrasound waves have been used for many years in numerous medical applications, for diagnostics and as therapy for various conditions. For instance, in medical imaging, ultrasonography is a widely used diagnostic technique that is used to see internal structures or organs of a patient, for instance muscles or tendons or for instance, in cardiovascular diagnostic technique to measure velocity of blood. Ultrasound is classically used in obstetric to examine a pregnant woman and provide reliable images of the child during pregnancy.

There are also various surgical applications based on ultrasound waves to solve cardiovascular disease (CVD). The main forms of CVD are stroke and coronary heart diseases (CHD). Coronary heart disease is the narrowing or blocking of coronary arteries, which results in a reduction of blood flow to heart muscle. Atherosclerosis is defined as the development of blockages due to presence of plaque. This is a gradual process starting with thickening of the arterial walls resulting in reduced blood flow and possible rapid total occlusion of the coronaries. In general, atherosclerotic lesions are localized in the intima of the artery wall.

Minimally interventional procedures are currently practiced restoring normal blood flow in the lumen of arteries or of the blood vessel that have full or partial blockage due to lesion. Minimally invasive mechanically based interventions concentrate on removing or de-bulking the lesion or blockage. Example of these are balloon angioplasty or percutaneous transluminal coronary angioplasty (PTCA), stenting, and rotational and directional atherectomy.

Most of these devices are similar from the point of view of the minimally invasive method used and require to be inserted into an artery either in the upper leg close to the inner thigh or the upper forearm, which allows direct access to the aorta and coronary arteries. A hollow tube, or catheter, is then manipulated to the location of the blockage, which acts as conduit for the main device. Generally, these devices aim to reduce the blockage in the arterial lumen by loading and permanently deforming the plaque.

These procedures present two main challenges. Firstly, the lesion must be capable of being crossed by a guidewire. This act as a guide rail to direct the device into the place. In some cases, it may be extremely difficult, if not impossible, to cross the lesion to reopen the blockage. These cases are known as chronic total occlusion (CTO) and are caused by advanced plaques, haemorrhaging and thrombosis and result in the total closure of vessel. If the lesion cannot be crossed due to total occlusion success levels of the procedure are greatly reduced.

Secondly, the mechanical properties of plaque which can be categorized from distensible to rigid. The more rigid lesions, also known as calcified plaques, have a great resistance to deformation or stenting and sometimes despite high pressure applied to the balloon it's not possible to deform them. In this case the stenting is also not possible. The shapes of lesions can be divided into following categories, such concentric and circular, such eccentric and circular, such eccentric and non-circular. These procedures, notably those that use dilatation balloon, work best with concentric lesions as the pressure is divided relatively evenly over the lesion. These complication, which are cause in part by the mechanical properties and the shapes of the lesions, affect the success rates of these interventional procedures.

In the light of the problem described, a device with capability of crossing all type of occlusions, including CTO, would be a major advantage. Ideally, a device that would be able to navigate in vascular vessels, which would specifically target diseased tissue and re-open blockages while causing little damage to the surrounding structure. Different solutions have been developed during 80 s to improve the success rates of these procedure, such as intravascular sonotherapy and high power, low frequency, therapeutic ultrasound. Intravascular sonotherapy is a prophylactic and therapeutic application of ultrasound, transmitted down a long wire waveguide. The ultrasound delivered has an operating frequency currently between 0.7-1.4 MHz. This technique has been used essentially after stenting to prevent restenosis. High power, low frequency, therapeutic ultrasound has been used to disrupt cardiovascular lesion. By choosing the right combination of frequency and amplitude, one can disrupt the plaque without damaging healthy tissue surrounding the lesion. It was conceived that this form of energy may be useful in disrupting cardiovascular lesions especially rigid calcified and fibrous plaques and would have advantages over standard procedures such as angioplasty or mechanical atherectomy. This technique and the device have been extensively described in the literature, for instance in U.S. Pat. Nos. 5,156,143 and 5,427,118.

An example of existing ultrasonic device 1 is illustrated in FIG. 1. Basically, ultrasonic device 1 for ultrasound angioplasty comprises an external power generator 2, a piezoelectric transducer 3, a horn 4 acting as principal amplificatory of displacement and a catheter 5 with a transmission wire 6, acting as waveguide, connected to the horn 4, and ending with a tip 7. The power generator 2 supplies the system with electrical energy that is required to produce ultrasonic energy, generally at the resonant frequency of the ultrasonic converter. The transducer 3 is generally made up of PZT crystals that convert the electrical energy of the generator 2 into mechanical displacements. The displacements achieved from transducers are still relatively small and too low for the intended application use, and therefore need to be amplified. This amplification is performed via an acoustic horn 4 or a waveguide (i.e. wire 6), which is attached to the end of the transducer.

The horn is characterized by its shape optimized to amplify the displacements. For instance, it is solid metal rod manufactured from material with high dynamic fatigue, high strength and low acoustic loss properties, such as titanium alloys. The amplification of displacement outputs is achieved by two methods. First, the surface area of the horn 4 reduces from the output face of the transducer to the horn output face. This reduction in surface area compresses the input waves resulting in a larger displacement at the output.

Secondly, horns can be manufactured to resonate at the frequency of the ultrasonic converter and are preferably designed to be exactly half the wavelength of the frequency.

Finally, in the goal to deliver the ultrasonic energy to the occlusion by traversing the tortuous vascular geometry, a waveguide, such the wire 6, is necessary. This waveguide is a part of the catheter 5 which is connected at the proximal end of the horn 4 and is connected with the tip 7 at the distal end. The ultrasonic energy is transmitted through the catheter 5, thanks the wire 6 acting as a waveguide, until the tip 7, to generate a displacement at the distal end of the tip 7. This displacement generates an acoustic energy which is deposited at the target of occlusion to destroy it.

When electrical energy is provided to the PZT stack, it vibrates longitudinally. The vibrations are then transmitted toward the tip 7 via the transmission wire 6 to produce a distal vibration of the tip 7. The horn 4 and the transmission wire 6, for instance a tapered transmission wire, allow amplification of the vibrations so that the vibration at the tip 7 are larger than the displacement of the PZT stack. Vibrations of the tip 7 generate an acoustic field 8 with an acoustic pressure shape centered on the tip 7.

The existing ultrasonic device as shown in FIG. 1 are mainly effective to treat concentric and circular lesions, as the acoustic pressure field generated by the tip is distributed relatively evenly over the lesion. The most important part of the acoustic pressure field is centered in front of the tip so that the existing solution provides the best results for treating concentric and circular lesions.

However, when lesions are located on one side of the vessel so that the region to be treated is not in front of the tip, the existing solution fails to provide satisfying results because the most part of the acoustic pressure field is not focused on the side of the wall where the lesions are located. Therefore, there is need to provide an improved ultrasonic vessel allowing to improve the treatment of the eccentric lesions located on the side of the vessel.

### SUMMARY OF THE DISCLOSURE

According to the invention, these aims are achieved by means of an ultrasonic device for generating an acoustic field in a lumen of a component of the cardiovascular system of a patient, in particular a blood vessel or a heart chamber, the device comprising a sonotrode, a transmission wire and a tip, the sonotrode comprising a transducer and a horn, the transducer being coupled to the horn, the transmission wire being coupled on one end with the horn and on the opposite end with the tip, so that when the transducer vibrates, said vibrations are amplified by the horn and transmitted to the transmission wire that displaces accordingly, said displacements of the transmission wire inducing vibrations of the tip generating an acoustic field from said tip, characterized in that the device comprises n sonotrode, n transmission wire, and at least one tip, n being superior or equal to two, and in that the device further comprises a control module being arranged for controlling the amplitude of displacement of each transmission wire so as to set up the emitted acoustic field generated from said tip.

In the present invention, the device comprises at least two sonotrodes and two transmission wires to provide a multi wires device. With the existing solution with one wire, it is necessary to use a diameter of the wire sufficiently large to transmit the necessary power to minimize losses that would result in overheating of the wire. This impacts the flexibility of the wire and limits access to tortuous path vessels where great flexibility is required. In the multi wire device according to the invention, the power is distributed in the n wires, so that it is possible to use a plurality of wires with a smaller diameter than when using a single wire of a large diameter like in the existing solution. This allows maintaining the flexibility of the device.

The device according to the present invention comprises a control module. The control module allows changing the shape of the acoustic field emitted from the tip, whether from one tip or from a plurality of tips. For instance, when it comes to treating eccentric lesion located on the side of the vessel, it is advantageous to have the most important part of the acoustic pressure field not centered in front of the tip but shifted or oriented or positioned on one side. The control module allows for instance shaping the emitted acoustic field toward one side of the vessel to treat in particular this region. This would keep or increase the effectiveness of treatment on the target region while protecting areas where treatment is not desired.

According to an embodiment, the device comprises m sonotrodes, m transmission wires and one single tip, all the transmission wires being coupled to said tip, m being superior or equal to two. In this embodiment, all the transmission wires are connected to a single tip.

When all transmission wires are connected to the same tip, the shape of the acoustic filed generated from the tip depends on the amplitude of displacement of each wire and on the phase of the displacement between the wires. FIGS. 2 through 5 represent a model with two wires connected on one tip, but the invention is not limited to this embodiment.

In FIGS. 2A and 2B, each wire oscillates in phase with the same amplitude of displacement around the point of rest R. The emitted acoustic field F1 is oriented in the axial direction, i.e. along the direction of the wires.

In FIGS. 3A and 3B, each wire oscillates in phase with a different amplitude of displacement around the point of rest R, the difference of amplitude d being induced by the control module. The tip follows a complex movement of rotation whose main direction is no longer along the main axis. The emitted acoustic field F2 is oriented mainly in the direction of the rotation of the tip.

In FIGS. 4A and 4B, each of the wires oscillates in opposition of phase (180° phase shift) with a different amplitude of displacement around the point of rest R, the difference of amplitude d and the phase shift being induced by the control module. The tip follows two main complex movements of rotation whose main directions are no longer along the main axis. The emitted acoustic field F3 is oriented mainly in the two directions of the rotation of the tip.

In FIGS. 5A and 5B, only one of the wires oscillates, with the other wire being non-oscillating and in fixed relation to the point of rest R. The difference of amplitude d of the oscillating wire is in relation to and alternates about the point of rest R. The tip follows a complex movement of rotation that is effectively about the non-oscillating wire at the point of rest R. The emitted acoustic field F4 is oriented mainly in the direction of the rotation of the tip.

The displacement of each transmission wire is triggered by the sonotrode coupled with said transmission wire via a horn. Each sonotrode is supplied with a power supply. In an embodiment, the control module controls the power supply, i.e. the electrical energy, provided to each sonotrode to control the amplitude of the displacement of each transmission wire. The control module is capable of balancing, in other word modulating, the power supply that is provided to the sonotrodes depending on the acoustic field that has to be generated from the tip. In other words, the control module acts as an upstream regulator of the ultrasounds waves emitted from the transducer.

In an embodiment represented in FIG. 17, the control module, composed of a microcontroller (DSP or FPGA for instance) is in charge of distributing and regulating the power, principally by adjusting the voltage and the frequency, to the sonotrodes. The microcontroller allows distributing the required power, through power stages, to the transducer of each sonotrode.

The control module can be connected to a user interface where the user can provide instructions to the control module, for instance to set the amplitude, the power, the shape of the emitted acoustic field. The user can also have a feedback on the status of the device in real time via this interface.

The control module comprise a computer program designed for controlling the parameters of the device. For instance, the program can comprise pre-configured menu or table comprising the appropriate combination of parameters (frequency, tension, displacement of the wire) depending on the requested shape of emitted acoustic field. In other words, the user provide the shape of the emitted filed required (via coordinates in a three dimension space, and/or pressure value), and the program adjusts automatically the parameters to provide the requested emitter acoustic field.

The control module can comprise tables for the n tip/n-transmission wire solution, and tables for the solution with at least two wires on a tip.

The tables could have predefined parameters, and predefined control sequences such as displacement modulation, PWM control, Wobulation control, or automatic rotation of the main direction of the sound field in different user-selected ranges of angles.

In a preferred embodiment, the control module controls the emitted acoustic field by controlling the power supply to each sonotrode. In particular, the control module controls the frequency and the tension applied to the transducer of each sonotrode, preferably continuously.

In another embodiment, a fixed tension is applied to each transducer and the control module controls the shape of the horn, for instance by applying torsion and/or compressions and/or release.

In another embodiment, a fixed tension is applied to each transducer, and the control module control the displacement of one or several masses placed on the sonotrodes to adjust the overall resonance frequency of the device in real time. According to an embodiment, the control module is arranged for providing a symmetrical and/or asymmetrical emitted acoustic field. In this embodiment, the control module manages the displacement of each transmission wire to provide either a symmetrical or an asymmetrical emitted acoustic field. For instance, if the device comprises two sonotrodes connected to one tip, and a symmetrical emitted field is required, the control module will ensure for an equal distribution of power supply to each of the sonotrode. On the contrary, if an asymmetrical emitted acoustic field is required, the control module will provide a biased distribution of energy supply in favor of one of the sonotrode, in other words one of the sonotrode will receive more power supply than the other one.

In some cases, it is advantageous to provide an asymmetrical emitted acoustic field if the thrombus or the stenosis are located on a side of the blood vessel for instance. Advantageously, the present device allows providing improved results in such cases compared to existing solutions.

In an embodiment, the control module is arranged for setting up the intensity and/or orientation of the emitted acoustic field. In particular, the control module can modulate the energy supply to provide a patterned emitted acoustic field or pressure field.

According to an embodiment, the control module allows setting up the emitted acoustic field in two dimensions defined in a plane (x,y).

In an embodiment, the control module allows setting up the emitted acoustic field in three dimensions defined in a space (x,y,z).

In an embodiment, the device comprises n sonotrodes, n transmission wires and n tips, each sonotrode being coupled to one transmission wire and one tip, n being superior or equal to two. In this embodiment, the acoustic field generated from the tip corresponds to the result of the displacements of each of the transmission wires, and is called emitted acoustic field. In other words, the emitted acoustic field generated at the tip is the sum of the acoustic field generated by each tip of each sonotrode. Each sonotrode provides a contribution to the emitted acoustic field. Each transmission wire vibrates longitudinally and/or laterally thereby generating vibrations of the tip that provide an acoustic field. The control module of the claimed device aims at controlling the displacement of each transmission wire to set up, i.e. modulate or regulate, the emitted acoustic field generated from the tip. Preferably, the displacement of the transmission wire are the displacement along the longitudinal axis of said transmission wire. The control module allows controlling the displacement of the wire so as to set up the acoustic field generated from the tip.

In one embodiment, the transducers of said sonotrodes are placed outside the lumen when said tip is positioned within said lumen. In many existing ultrasonic device, the transducer is the source of ultrasonic waves and is placed within the lumen during operation.

In an embodiment, the center of the tip is free from transmission wire, said center of the tip further comprise a traversing hole for passing a guidewire. In many medical applications, for instance cardiology applications, angioplasty frequently comprises the use of use of ultrasound followed or associated with the use of a guidewire passing though the tip to mechanically disrupt the thrombus or the stenosis. Generally, the guidewire needs to pass though the center of the tip to ensure an efficient positioning with the lumen of the vessel. The device according to the present embodiment facilitates treatment ultrasound and guidewire based treatment.

According to an embodiment, wherein the tip has a spherical shape. Alternatively, the tip can have a cylindrical shape, ovoid, with concave cavity for instance like a golf ball, hemi spherical, spherical with lobes fixed thereon, ovoid with a cavity, cylindrical with a cavity in the body of the cylinder, preferably a spherical shape. Preferably, the choice of the type of tip geometry will be according to the type of thrombus or stenosis (position, hardness) and according to the choice of the control mode (PWM, wobulation, phase shift) privileged for the procedure.

In an embodiment, the tip has a maximal diameter comprised between 0.5 mm and 5 mm, preferably between 1.25 mm and 2.5, mm, more preferably between 1.5 and 2.25 mm.

According to an embodiment, the transducer is a chosen among piezoelectric transducer, for instance PZT, or comprises a magnetostrictive material. Preferably, the transducer is a PZT transducer comprising a stack of piezo electric material.

In an embodiment, the device further comprising a power generator for supplying energy to said sonotrodes. In particular, the device can comprise one generator for all the sonotrodes. Or the device can comprise a generator per sonotrode.

According to an embodiment, each sonotrode is supplied with a power comprises between 5 and 300 watts, preferably between 8 watts and 100 watts, more preferably between 10 watts and 50 watts.

In an embodiment, each transmission wire is received in a catheter, said tip exiting the catheter.

In the present invention, the terms “acoustic field” and “pressure field” are interchangeable.

In the present invention, the term transducer defines an element that converts the electrical energy into ultrasonic energy.

In the present embodiment, the terms “shape of the emitted acoustic or pressure field” define the dimensions of said field. In other word, the word shape is used to describe the distribution of the acoustic field around the tip.

### BRIEF DESCRIPTION OF THE DRAWINGS

The invention will be better understood with the aid of the description of an embodiment given by way of example and illustrated by the figures, in which:

FIG. 1 shows a view of an existing device of the prior art;

FIGS. 2A through 6A show a first embodiment of the device according to the invention;

FIG. 6B shows a second embodiment of the device according to the invention;

FIG. 7 shows the tip of the device according to the first and second embodiments;

FIGS. 8A, 8B, and 9 show the distribution of the acoustic field of the tip of the device according to the first embodiment;

FIG. 10 shows the evolution of the angle of the maximum of the absolute pressure generated from the tip in the first embodiment of the device according to the invention;

FIG. 11A shows a third embodiment of the device according to the invention;

FIG. 11B shows a fourth embodiment of the device according to the invention;

FIG. 12 shows the tip of the device according to the third and fourth embodiments;

FIGS. 13A, 13B, and 14 show the distribution of the acoustic field of the tip of the device according to the third embodiment;

FIG. 15 shows the evolution of the angle of the maximum of the absolute pressure generated from the tips in the third embodiment of the device according to the invention;

FIG. 16 shows a fifth embodiment of the device according to the present invention;

FIGS. 17A and 17B shows the distribution of the acoustic field of the tip of the device according to the fifth embodiment;

FIG. 18 shows the control module of the device according to the first and third embodiments of the device according to the invention; and

FIGS. 19A through 19F shows various embodiments of the tip of the device according to the invention.

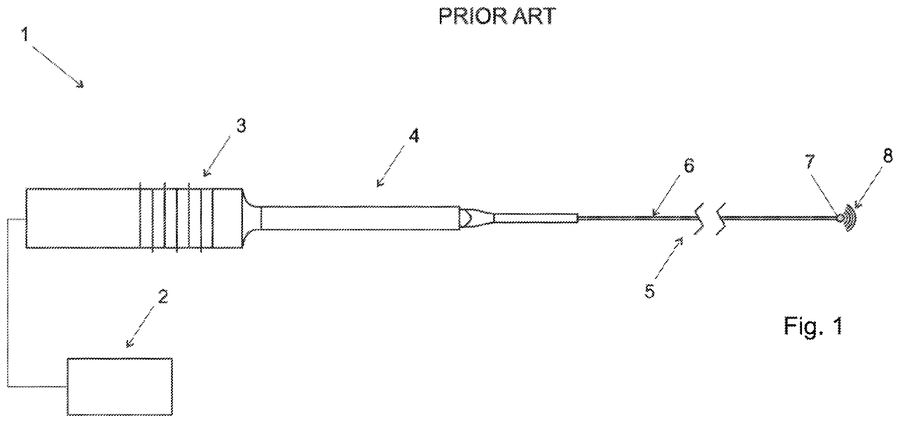

### DETAILED DESCRIPTION OF THE FIGURES

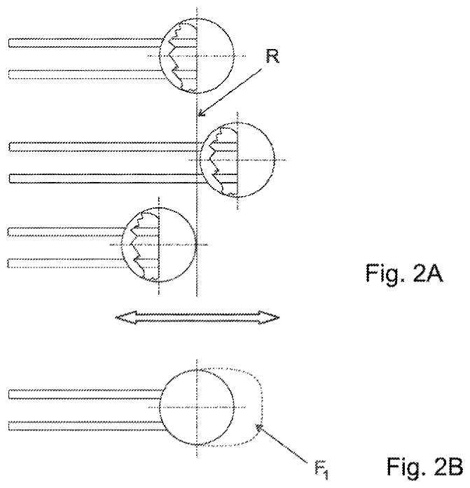

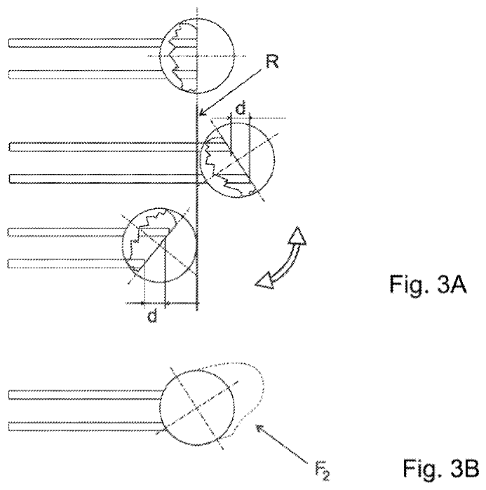

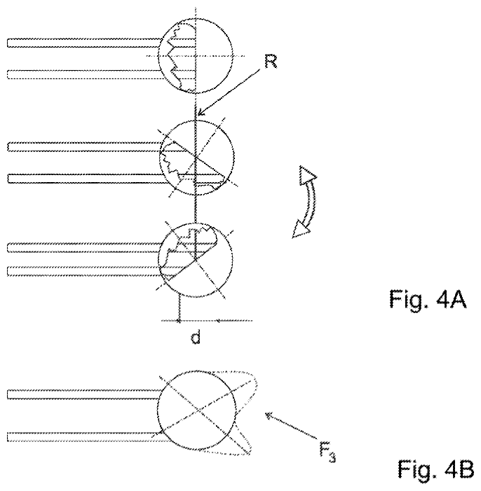

FIGS. 2A through 19F represent several embodiments of the present invention, but the invention is not limited to the disclosed embodiments.

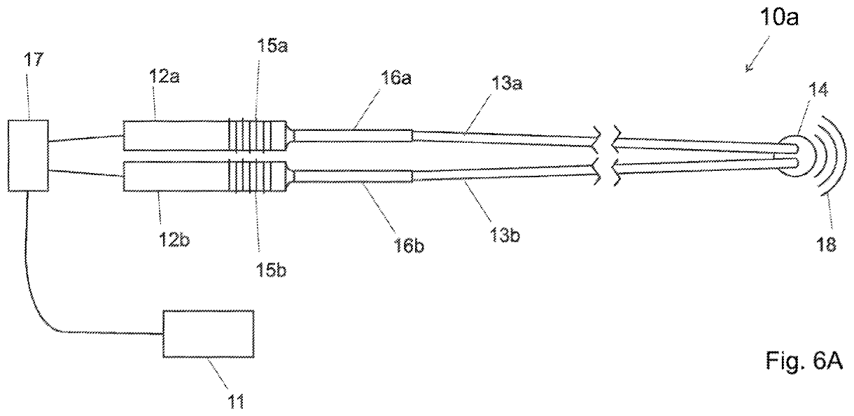

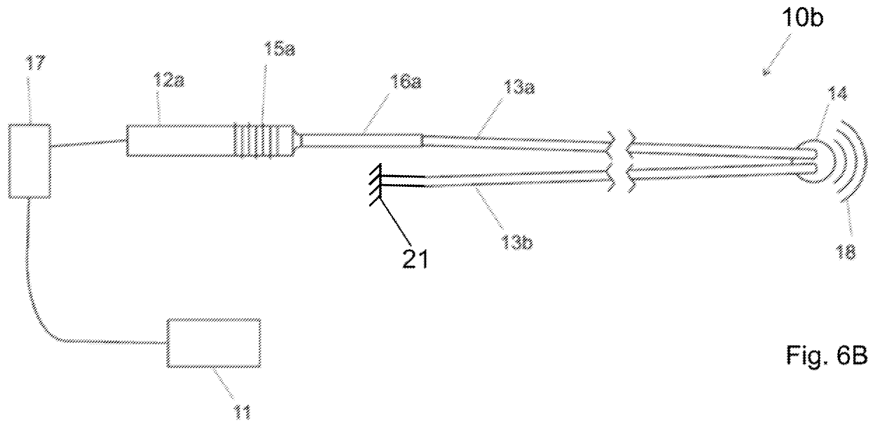

FIG. 6A represents a device 10a according to a first embodiment. In this embodiment, the device 10a comprises power generator 11 providing electrical energy to the device 10a. The device 10a further comprises two sonotrodes 12 a,b, each sonotrode 12 a,b being connected to one transmission wire 13 a,b. In this embodiment, all the transmission wires 13a,b are connected to the same single tip 14. Each sonotrode 12a,b comprises one transducer 15a,b that is connected to a horn 16a,b.

In this first embodiment, the transducers 15a,b are PZT stack. The transmission wires 13a,b have a diameter of 0.5 mm and the tip 14 have a diameter of 2 mm. The tip 14 and the wire 13a,b are made with TiAl₆V₄ but the invention is not limited to this material, for instance Aluminium could also be used.

The control module 17 allows controlling the electrical energy provided to each transducer 15a,b of each sonotrode 12a,b to set up the emitted acoustic field 18.

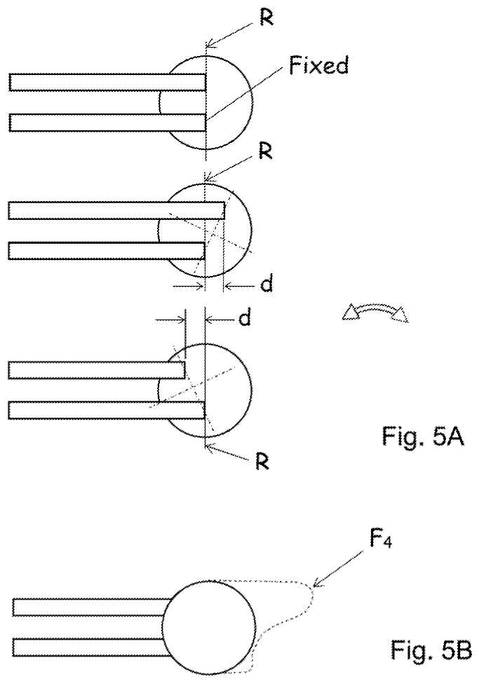

FIG. 6B represents a device 10b according to a second embodiment of the disclosure. The device 10b includes some of the same components and attributes as the device 10a, some of which are identified by same-labeled reference characters. As with device 10a, the device 10b comprises power generator 11 providing electrical energy to the device 10b. However, the device 10b comprises only one sonotrode 12a, which is connected to one transmission wire 13a. The second wire 13b is mechanically fixed to an anchor point 21, for example, by clamping, pinning, screwing or welding to a frame (not depicted) that supports the sonotrode 12a or a housing (not depicted) that houses the sonotrode 12a. The mechanically fixed anchor point 21 holds the wire 13b in fixed relationship with the point of rest Rat the tip 14, thereby effecting the motion and emitted acoustic field F4 described at FIGS. 5A and 5B.

The device 10a can also be made to function as depicted in FIGS. 5A and 5B. By not powering sonotrode 12b of device 10a, the effect is the same as mechanically fixing the transmission wire 13b. Herein, to “energize” a sonotrode is to supply the sonotrode with an alternating voltage or current. Accordingly, a sonotrode is “not energized” when no voltage or current is supplied, or when a DC voltage or current is supplied.

For the devices 10a and 10b, wires 13a,b are connected to a single tip 14. The transducers 15a,b are PZT stacks. The transmission wires 13a,b have a diameter of 0.5 mm and the tip 14 have a diameter of 2 mm. The tip 14 and the wire 13a,b are made with TiAl₆V₄ but the invention is not limited to this material, for instance Aluminium could also be used.

The control module 17 allows controlling the electrical energy provided to each transducer 15a,b of each sonotrode 12a,b to set up the emitted acoustic field 18.

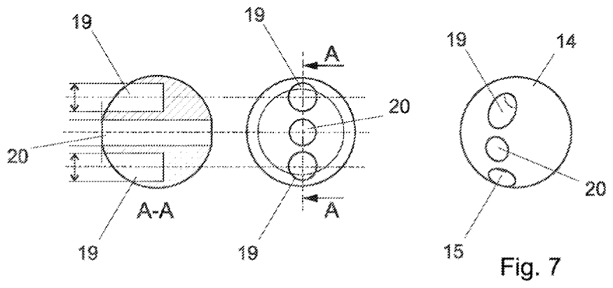

FIG. 7 shows the tip 14 of the devices 10a and 10b. The tip 14 comprises two blind holes 19 design for receiving the transmission wires 13a,b. The tip 14 further comprises a traversing hole 20 for passing a guidewire (not represented in the figures).

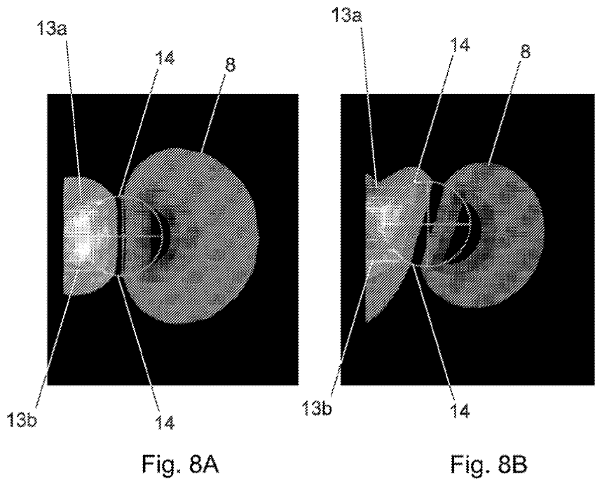

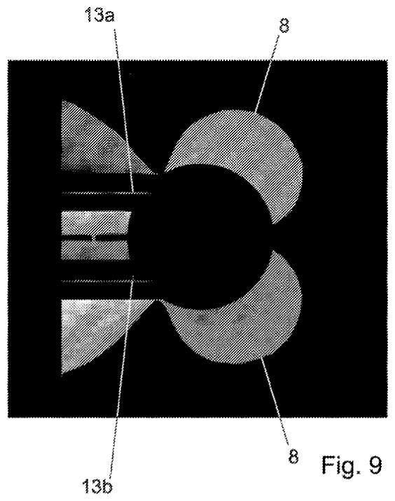

FIGS. 8A, 8B, and 9 represent the distribution of the acoustic field 8 in operation for the first embodiment.

FIG. 8A represents a case where the each wire receive the amount of energy from the control module 17, to provide the same amplitude of displacement on the wires and where the wires oscillate in phase. The emitted acoustic pressure 8 is symmetrical and centered around an axis parallel to the longitudinal axis of the transmission wires 13a,b.

FIG. 8B represents a case where there is a ratio of ten (10) between the transmission wire 13a,b, said ratio being induced by the control module 17. In other words, the amplitude of displacement along the longitudinal axis of one transmission wire 13a is 50 microns, whereas the other transmission wire 13b is 5 microns and where the wires oscillate in phase. Consequently the emitted acoustic field 8 is asymmetric and oriented toward the transmission wire with the larger displacement. By selecting the wire on which a difference of amplitude is applied, it is possible to orient and to control in the plane (XY) the asymmetry of the acoustic field. In this embodiment, it is possible, when the device is placed in a vessel, to position the emitted acoustic field for instance toward a side of the vessel, to provide a major contribution of the acoustic field on the side of the vessel, and a minor contribution on the other side of the vessel. This can improve the treatment of an eccentric lesion by increasing the efficiency of the treatment on the area of the vessel where the treatment is needed and to protect, by limiting the acoustic field, the area where the treatment is not necessary.

FIG. 9 represents a case where each wire oscillates in opposition of phase (180° phase shift), with a displacement around the point of rest. The control module 17 ensures that each transmission wire receives the amount of energy to provide the same amplitude of displacement but the control module 17 induces a phase shift of 180°. As shown in FIG. 9, the emitted acoustic field is divided in two main contributions each oriented toward the side of the vessel when the tip 14 is placed in a vessel. Therefore, in this embodiment, it is possible to target occlusion located on both sides of the vessel and to improve the treatment of complex eccentric lesions.

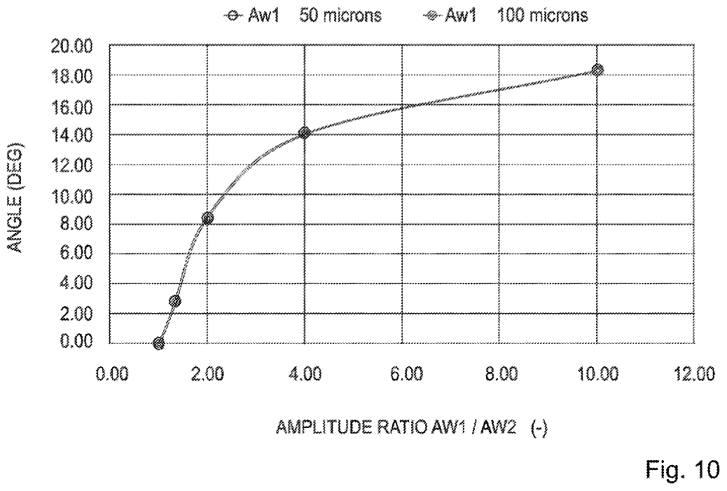

FIG. 10 represents the angle of the maximum absolute pressure point on the tip distal face depending on the amplitude ration between the transmission wires. The plot shows that at a ratio of 10, it is possible to shift maximum pressure point 18° with respect to the position of said maximum pressure point when the ratio is 1. Therefore, this plot demonstrates that it is possible to control the shape (i.e., the orientation) of the emitted pressure field by controlling the relative displacement of the transmission wires of the device. This plot also demonstrates that this behavior (i.e., the orientation of the maximum absolute pressure point) is not impacted by the initial choice of the amplitude of displacement applied on the wires (as example 100 microns instead of 50 microns), but only by the choice of the amplitude ratio between the transmission wires. On the other hand, the intensity of the field is dependent on the choice of the amplitude of displacement. Greater displacement amplitude induces a higher max pressure value.

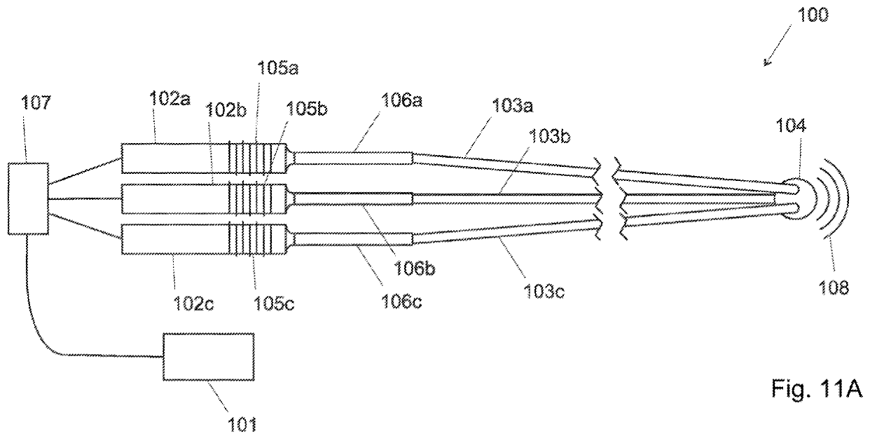

FIG. 11A represents a third embodiment of the device 100a. In this embodiment, the device 100a comprises power generator 101 providing electrical energy to the device 100a. The device 100 further comprises three sonotrodes 102a,b,c each sonotrode 102a,b,c being connected to a transmission wire 103a,b,c that leads to a tip 104 at the distal end of said transmission wire 103a,b,c. The three transmission wires 103a,b,c are connected to the same tip 104. Each sonotrode 102a,b,c comprises a transducer 105a,b,c that is connected to a horn 106a,b,c.

In this third embodiment, the transducers 105a,b,c are PZT stack. The transmission wires 103a,b,c have a diameter of 0.5 mm and the tip 104 have a diameter of 2 mm. The tip 104 and the wire 103a,b,c are made with TiAl₆V₄ but the invention is not limited to this material, for instance Aluminium could also be used.

The device 100a further comprises a control module 107 arranged for controlling the amplitude of displacement of the transmission wires 103a,b,c along the longitudinal axis. The amplitude of displacement of the transmission wire 103a,b,c depends on the energy supply provided to by the power generator 101 to the transducers 105a,b,c. The amplitudes of vibrations of the PZT stack is correlated to the electrical energy provided by the power generator. In the present embodiment, the control module 107 allows controlling the electrical energy provided to each transducer 105a,b,c of each sonotrode 102a,b,c to set up the emitted acoustic field 108.

FIG. 11B represents a device 100b according to a fourth embodiment of the disclosure. The device 100b includes some of the same components and attributes as the device 100a, some of which are identified by same-labeled reference characters. As with device 100a, the device 100b comprises power generator 101 providing electrical energy to the device 100b. However, the device 100b comprises only two sonotrodes 102a,b which are connected to transmission wires 103a,b respectively. The third wire 103c is mechanically fixed to an anchor point 111, for example, by clamping, pinning, screwing or welding to a frame (not depicted) that supports the sonotrode 102a,b, or a housing (not depicted) that houses the sonotrode 102a,b. The mechanically fixed anchor point 111 holds the wire 103c in fixed relationship with the point of rest of the tip 104. The resultant effect is a motion and emitted acoustic field akin to that described at FIGS. 5A and 5B.

The device 100a of FIG. 11A can also be made to function as depicted in FIGS. 5A and 5B. By not energizing sonotrode 102c of device 100a, the effect is the same as mechanically fixing the transmission wire 103c.

For devices that utilize three or more sonotrodes, the number of wires 103 that are mechanically fixed or not energized is not limited to a single sonotrode. Rather, the number of wires 103 that are mechanically fixed or not energized may range from 1 to n−1, where n is the total number of wires 103 connected to the tip 104.

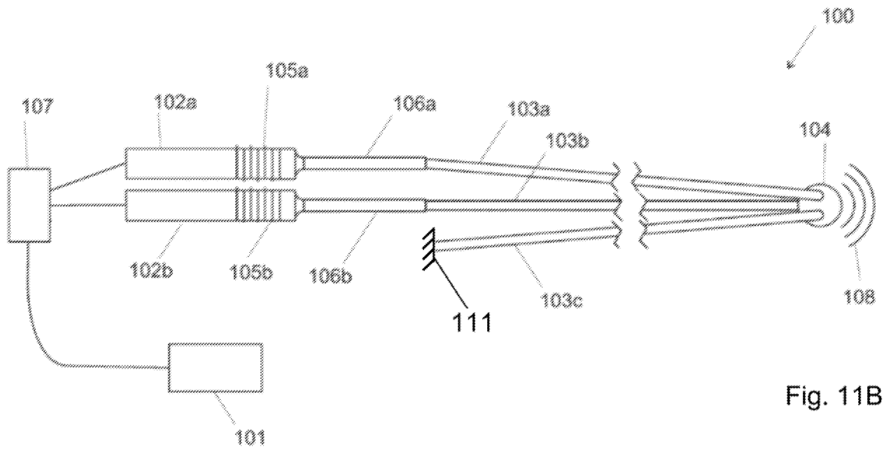

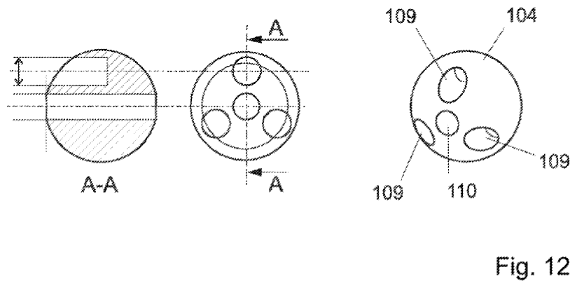

FIG. 12 illustrates the tip 104 of the device 100 according to the third and fourth embodiments. The tip 104 comprises three blind holes 109 design for receiving the transmission wires 103a,b, c. The tip 104 further comprises a traversing hole 110 for passing a guidewire (not represented in the figures).

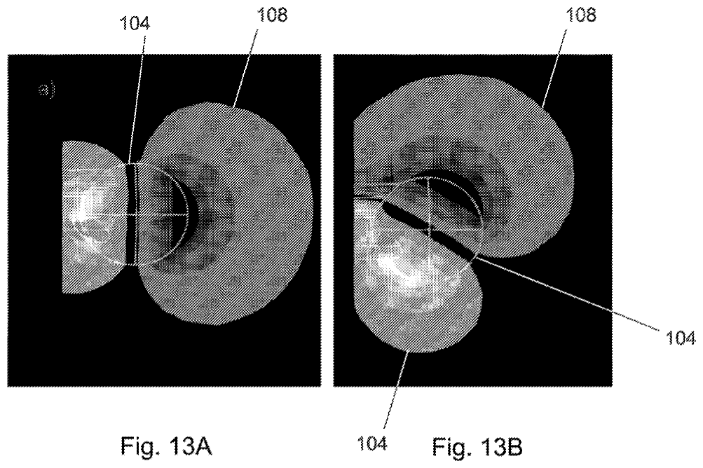

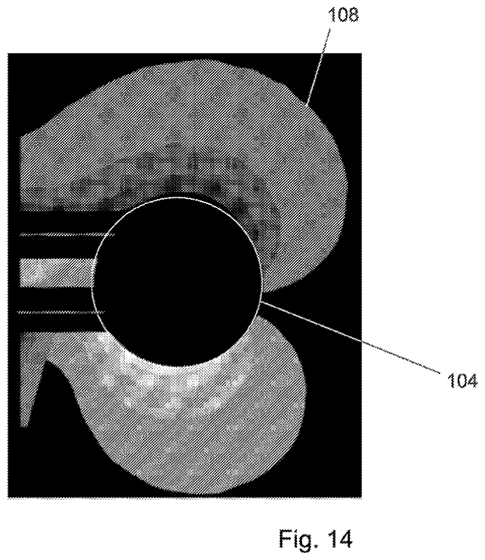

FIGS. 13A, 13B, and 14 represent the distribution of the acoustic field 108 in operation for the third embodiment.

FIG. 13A represents a case where the each wire receive the amount of energy from the control module 107 to provide the same amplitude of displacement on the wires, and where the wires oscillate in phase. The emitted acoustic pressure 108 is symmetrical and centered around an axis parallel to the longitudinal axis of the transmission wires 103a,b, c.

FIG. 13B represents a case where there is a ratio of ten (10) between the transmission wires 103a,b,c said ratio being induced by the control module 107. In particular, two transmission wires receive the amount of energy to provide the same amplitude of displacement on each one, the third one receive an amount of energy to provide an amplitude of displacement ten (10) times less than the two others transmission wires. In other words, the amplitude of displacement along the longitudinal axis of two transmission wires 103a,b is 50 microns, whereas the other transmission wire 103c is 5 microns, and where the three wires oscillate in phase. Consequently the emitted acoustic field 108 is asymmetric and oriented toward the transmission wires with the larger displacement. By selecting the wires on which a difference of amplitude is applied, it is possible to orient and to control in the space (XYZ) the asymmetry of the acoustic field. In this embodiment, it is possible when the device is placed in a vessel, to position the emitted acoustic field for instance toward a side of the vessel, to provide a major contribution of the acoustic field on the side of the vessel, and a minor contribution on the other side of the vessel. This can improve the treatment of an eccentric lesion by increasing the efficiency of the treatment on the area of the vessel where the treatment is needed and to protect, by limiting the acoustic field, the area where the treatment is not necessary.

FIG. 14 represents a case where one wire oscillates in opposition of phase (180° phase shift) of the two others, with a displacement around the point of rest. The control module 107 ensure that each transmission wire receives the amount of energy to provide the same amplitude of displacement on each wire but the control module 107 induces a phase shift of 180° between one of the wire and the two others. As shown in FIG. 14, the emitted acoustic field is divided in two main contributions. By selecting the wire on which the phase shift is applied, it is possible to orient in the space (xyz) these two contributions. Therefore, in this embodiment, it is possible to target occlusion located on both sides of the vessel and to improve the treatment of complex eccentric lesions.

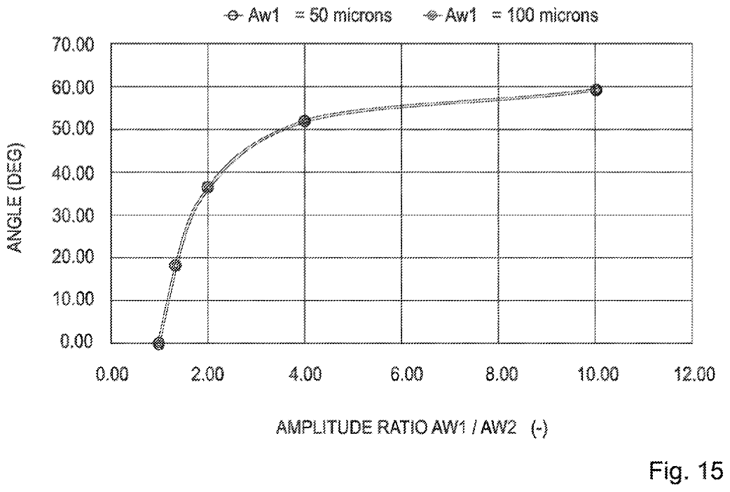

FIG. 15 represents the angle of the maximum absolute pressure point on the tip distal face depending on the amplitude ration between the transmission wires. The plot shows that a ratio of 10, it is possible to shift maximum pressure point of 60° with respect to the position of said maximum pressure point when the ratio is 1. Therefore, this plot demonstrate that it is possible to control the shape (i.e., the orientation) of the emitted pressure field by controlling the relative displacement of the transmission wires of the device. This plot demonstrates also that this behavior (i.e. the orientation of the maximum absolute pressure point) is not impacted by the initial choice of the amplitude of displacement applied on the wires (as example 100 microns instead of 50 microns), but only by the choice of the amplitude ratio between the transmission wires. On the other hand, the intensity of the field is dependent on the choice of the amplitude of displacement. Greater displacement amplitude induces a higher max pressure value.

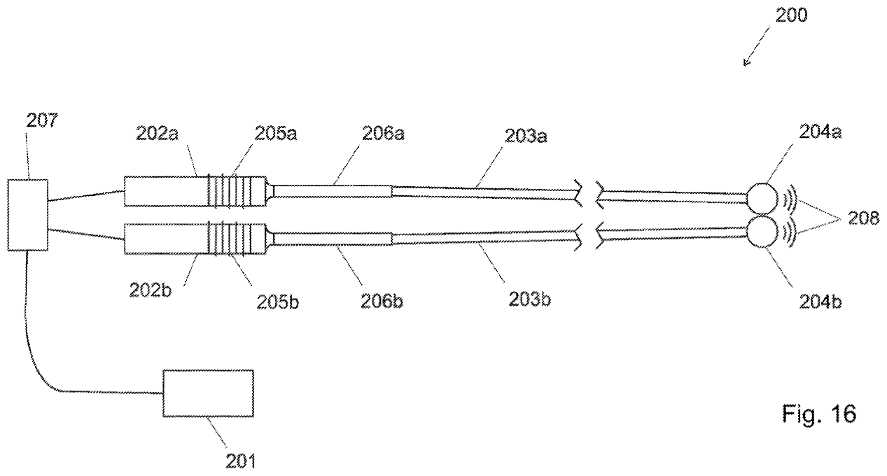

A fifth embodiment of the device is represented in FIG. 16. In this embodiment, the device 200 comprises power generator 201 providing electrical energy to the device 200. The device 200 further comprises two sonotrodes 202a,b each sonotrode 202a,b being connected to a transmission wire 203a,b that leads to a tip 204a,b at the distal end of said transmission wire 203a,b. In this embodiment, each transmission wire 203a,b is connected to one tip 204a,b. Each sonotrode 202a,b comprises a transducer 205a,b that is connected to a horn 206a,b.

In this fifth embodiment, the transducers 205a,b are PZT stacks. The transmission wires 203a,b have a diameter of 0.5 mm and the tip 204a,b have a diameter of 0.9 mm. The tips 204a,b and the wires 203a,b are made with TiAl₆V₄ but the invention is not limited to this material, for instance Aluminium could also be used.

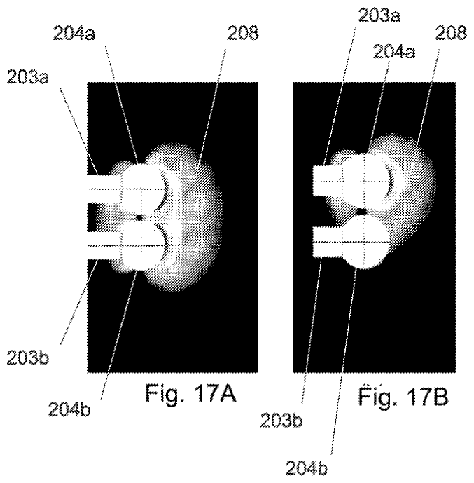

FIGS. 17A and 17B represent the shape of the acoustic field of the device according to the fifth embodiment in operation. In FIG. 17A, both sonotrodes 202a,b received the amount of electrical energy from the control module 207, to provide the same amplitude of displacement on the wires and where the wires oscillate in phase so that the acoustic fields generated from the tips 204a, b are similar. Therefore, each sonotrode 202a,b contributes equally to the generated acoustic field 208 generated from the tips 204a,b.

In FIG. 17B, the control module 207 is set up in favor of one sonotrode 202a so that said sonotrode 202a receives a bigger amount of electrical energy than the other one receives. As a result, the emitted acoustic filed 208 corresponds more to the acoustic field contribution of the sonotrode 202a, that receives a higher amount of the electrical energy, than the acoustic field contribution from the other sonotrode that receives a lower amount of electrical energy. In this embodiment, it is possible, when the device is placed in a vessel, to position the emitted acoustic field for instance toward a side of the vessel, to provide a major contribution of the acoustic field on the side of the vessel, and a minor contribution on the other side of the vessel. This can improve the treatment of an eccentric lesion by increasing the efficiency of the treatment on the area of the vessel where the treatment is needed and to protect, by limiting the acoustic field, the area where the treatment is not necessary.

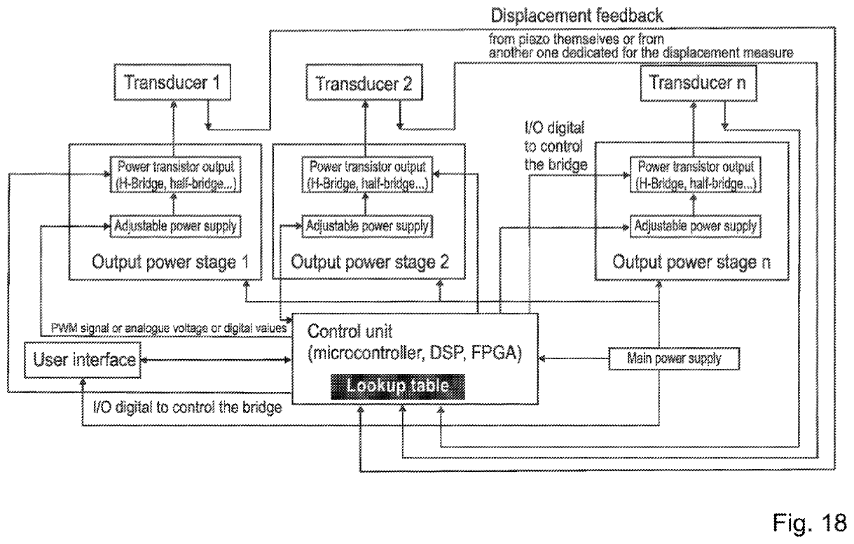

FIG. 18 is a chart representing the control module 17,107,207 used in the device according to the disclosed embodiments 10a,b, 100a,b, 200. The controller 17, 107, 207 controls the energy supply to each sonotrode so as to set up de emitted acoustic field 18, 108, 208. The controller is composed of a central unit equipped of a microcontroller or DSP or FPGA. This central unit controls through n outputs stage (n corresponding of the number of transducer), the frequency and the voltage to apply (i.e. the correct amount of energy) to each transducer in the goal to obtain the desired amplitude of displacement on each wire. A feedback on the amplitude value of displacement comes from each transducer and is interpreted by the central unit which can, by a control loop, control in real time the amount of energy to give to each transducer. A lookup table contains all the parameters necessary for the proper functioning of the device, depending on the case of n wires on one tip, or n wires and n tip, or the geometry of the tip as well as the safety parameters. The central unit is connected to a user interface that permits the user to control the device and to have feedback information on the status of the system.

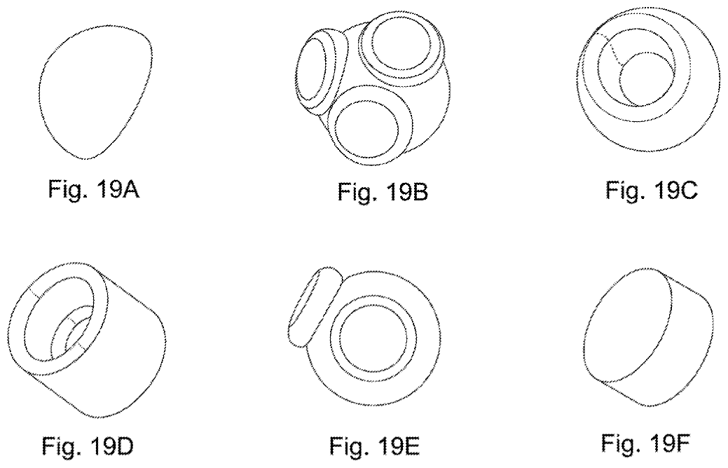

FIGS. 19A through 19F shows some embodiments of the tip 14, 104, 204 that can be used in the device according to the present invention. The shape of the tip 14, 104,204 can be: Spherical (FIGS. 6 and 11); hemispherical (FIG. 19A); spherical with lobes fixed thereon (FIGS. 19B and 19E); ovoid with a cavity (FIG. 19C); cylindrical with a cavity in the body of the cylinder (FIG. 19D); or cylindrical (FIG. 19F).

Advantageously, the shape of the tip can be chosen depending on the shape of the emitted acoustic field, according to the type of area to treat and according to the choice of the control mode privileged for the procedure (PWM, Wobulation, phase shift).

### REFERENCES OF THE FIGURES

1 Device according to the prior art
2 Power generator
3 Piezoelectric transducer
4 Horn
5 Catheter
6 Transmission wire
7 Tip
8 Acoustic field
10a Device according to a first embodiment with two transmission wires on one tip
10b Device according to a second embodiment with two transmission wires on one tip, one wire is fixed
11 Power generator
12a,b Sonotrode
13a,b Transmission wire
14 Tip
15a,b Transducer
16a,b Horn
17 Control module
18 Emitted acoustic field
19 Blind hole
20 Traversing hole
21 Mechanically fixed anchor point
100a Device according to a third embodiment with three transmission wires on one tip
100b Device according to a fourth embodiment with three transmission wires on one tip, one wire is fixed
101 Power generator
102a,b,c Sonotrode
103a,b,c Transmission wire
104 Tip
105a,b,c Transducer
106a,b,c Horn
107 Control module
108 Emitted acoustic field
109 Blind hole
110 Traversing hole
111 Mechanically fixed anchor point
200 Device according to a fifth embodiment
201 Power generator
202a,b Sonotrode
203a,b Transmission wire
204a,b Tip
205a,b Transducer
206a,b Horn
207 Control module
208 Emitted acoustic field

What is claimed is:

1. Ultrasonic device for generating an acoustic field in a lumen of a component of a cardiovascular system of a patient, in particular a blood vessel or a heart chamber, the device comprising a sonotrode, a transmission wire and a tip,
said sonotrode comprising a transducer and a horn, the transducer being coupled to the horn,
the transmission wire being coupled on one end with the horn and on an opposite end with the tip,
so that when the transducer vibrates, said vibrations are amplified by the horn and transmitted to the transmission wire that displaces accordingly, said displacements of the transmission wire inducing vibrations of the tip generating an acoustic field from said tip,
characterized in that the device comprises n sonotrodes, n transmission wires, and at least one tip, n being superior or equal to two, and in that the device further comprises a control module being arranged for controlling an amplitude of displacement of each transmission wire so as to set up an emitted acoustic field generated from said tip.

2. Ultrasonic device according to claim 1, wherein the device comprises m sonotrodes, m transmission wires, and one single tip, all the transmission wire being coupled to said tip, m being superior or equal to two.

3. Ultrasonic device according to claim 1, wherein the device comprises n sonotrodes, n transmission wires and n tips, each sonotrode being coupled to one transmission wire and one tip, n being superior or equal to two.

4. Ultrasonic device according to claim 1, wherein the device comprises n sonotrodes, n transmission wires and a maximum of n−1 tips, n being superior or equal to two, so that at least two of the n transmission wires are connected to a same of said n−1 tips.

5. Ultrasonic device according to claim 1, wherein the transducers of said sonotrodes are placed outside a lumen of a component of a cardiovascular system of a patient when said tip is positioned within said lumen.

6. Ultrasonic device according to claim 1, wherein the control module controls a power supply provided to each sonotrode to control the amplitude of displacement of each transmission wire.

7. Ultrasonic device according to claim 1, wherein the control module allows setting up the emitted acoustic field in two dimensions defined in a plane.

8. Ultrasonic device according to claim 1, wherein the control module allows setting up the emitted acoustic field in three dimensions defined in a space.

9. Ultrasonic device according to claim 1, wherein the control module is arranged for providing one or both of a symmetrical emitted acoustic field and an asymmetrical emitted acoustic field.

10. Ultrasonic device according to claim 1, wherein the control module is arranged for setting up at least one of an intensity and an orientation of the emitted acoustic field.

11. Ultrasonic device according to claim 1, wherein a center of the tip is free from transmission wire, said center of the tip further comprises a traversing hole for passing a guidewire.

12. Ultrasonic device according to claim 1, wherein the tip has a shape chosen among spherical shape, hemispherical, spherical with lobes fixed thereon, ovoid with a cavity, cylindrical with a cavity in a body thereof, or cylindrical.

13. Ultrasonic device according to claim 1, wherein the tip has a maximal diameter comprised between 0.5 mm and 5 mm.

14. Ultrasonic device according to claim 1, wherein the transducer is one of a piezoelectric transducer and a magnetostrictive material.

15. Ultrasonic device according to claim 1, the device further comprising a power generator for supplying energy to said sonotrodes.

16. Ultrasonic device according to claim 1, wherein each sonotrode is supplied with a power comprises between 5 and 300 watts.

17. Ultrasonic device according to claim 1, wherein each transmission wire is received in a catheter, said tip exiting the catheter.
